(19) United States
(12) Patent Application Publication
(10) Pub. No.: US 2024/0164785 A1
(43) Pub. Date: May 23, 2024

(54) ROTATABLE ULTRASOUND CATHETER FOR DETECTING LEFT ATRIAL APPENDAGE CLOSURE DEVICE LEAKS

(71) Applicant: BOSTON SCIENTIFIC SCIMED, INC., MAPLE GROVE, MN (US)

(72) Inventors: Joshua Mark Inouye, Brooklyn Park, MN (US); Jerry Wang, Edina, MN (US); James M. Anderson, Corcoran, MN (US)

(73) Assignee: BOSTON SCIENTIFIC SCIMED, INC., MAPLE GROVE, MN (US)

(21) Appl. No.: 18/516,196

(22) Filed: Nov. 21, 2023

Related U.S. Application Data
(60) Provisional application No. 63/427,696, filed on Nov. 23, 2022.

(51) Int. Cl. A61B 17/12; A61B 8/06; A61B 8/12

(52) U.S. Cl. CPC A61B 17/12122; A61B 8/06; A61B 8/12; A61B 17/12172; A61B 2017/1205

(57) ABSTRACT

Implanting a left atrial appendage closure (LAAC) device within a patient's left atrial appendage (LAA) may include advancing an assembly to a position proximate the patient's LAA. The assembly includes an LAAC device releasably secured to an LAAC delivery catheter, and one or more ultrasound transducers disposed relative to the LAAC delivery catheter. Once the LAAC device is deployed, the ultrasound transducers may be rotated relative to the LAAC device to look for leaks between the LAAC device and the LAA. In some cases, the LAAC device may be repositioned relative to the LAA when excessive leaks are found via ultrasound.

### CROSS-REFERENCE TO RELATED APPLICATIONS

This application claims the benefit of U.S. Provisional Patent Application Ser. No. 63/427,696 filed Nov. 23, 2022, the disclosure of which is incorporated herein by reference.

### TECHNICAL FIELD

The disclosure relates generally to medical devices and more particularly to medical devices that are adapted for detecting leaks around a left atrial appendage closure device.

### BACKGROUND

The left atrial appendage is a small organ attached to the left atrium of the heart. During normal heart function, as the left atrium constricts and forces blood into the left ventricle, the left atrial appendage constricts and forces blood into the left atrium. The ability of the left atrial appendage to contract assists with improved filling of the left ventricle, thereby playing a role in maintaining cardiac output. However, in patients suffering from atrial fibrillation, the left atrial appendage may not properly contract or empty, causing stagnant blood to pool within its interior, which can lead to the undesirable formation of thrombi within the left atrial appendage.

Thrombi forming in the left atrial appendage may break loose from this area and enter the blood stream. Thrombi that migrate through the blood vessels may eventually plug a smaller vessel downstream and thereby contribute to stroke or heart attack. Clinical studies have shown that the majority of blood clots in patients with atrial fibrillation originate in the left atrial appendage. As a treatment, medical devices have been developed which are deployed to close off the left atrial appendage. Of the known medical devices and methods, each has certain advantages and disadvantages. There is an ongoing need to provide alternative medical devices as well as alternative methods for manufacturing and using medical devices.

### SUMMARY

This disclosure provides design, material, manufacturing method, and use alternatives for medical devices. An example may be found in a method of implanting a left atrial appendage closure (LAAC) device within a patient's left atrial appendage (LAA). The method includes advancing an assembly to a position proximate the patient's LAA, the assembly including an LAAC device releasably secured to an LAAC delivery catheter, and one or more ultrasound transducers disposed relative to the LAAC delivery catheter. The method includes deploying the LAAC device within the LAA and then rotating the ultrasound transducers relative to the LAAC device to look for gaps and/or leaks between the LAAC device and the LAA.

Alternatively or additionally, the method may further include repositioning the LAAC device in response to detecting gaps and/or leaks.

Alternatively or additionally, the one or more ultrasound transducers may be part of an ultrasound catheter.

Alternatively or additionally, the ultrasound catheter may be steerable.

Alternatively or additionally, the method may further include steering the ultrasound catheter to get a better view of any possible leaks between the LAAC device and the LAA.

Alternatively or additionally, rotating the one or more ultrasound transducers may include rotating the ultrasound catheter around the LAAC delivery catheter.

Alternatively or additionally, rotating the one or more ultrasound transducers may include rotating the ultrasound catheter in place relative to the LAAC delivery catheter.

Alternatively or additionally, the one or more ultrasound transducers may be disposed on the LAAC delivery device.

Alternatively or additionally, rotating the one or more ultrasound transducers may include rotating the LAAC delivery device.

Another example may be found in an assembly adapted for implanting a left atrial appendage closure (LAAC) device within a patient's left atrial appendage (LAA). The assembly includes an LAAC device releasably secured to an LAAC delivery catheter and one or more ultrasound transducers disposed relative to the LAAC delivery catheter.

Alternatively or additionally, the one or more ultrasound transducers may be part of an ultrasound catheter.

Alternatively or additionally, the ultrasound catheter may be steerable.

Alternatively or additionally, the ultrasound catheter may be adapted to rotate around the LAAC delivery catheter.

Alternatively or additionally, the ultrasound catheter may be adapted to rotate in place relative to the LAAC delivery catheter.

Alternatively or additionally, the one or more ultrasound transducers may be disposed on the LAAC delivery catheter.

Alternatively or additionally, the LAAC delivery device may have a releasable connection between the LAAC delivery catheter and the LAAC device, and the releasable connection may be adapted to permit relative rotation therebetween without releasing the LAAC device.

Another example may be found in an assembly adapted for implanting a left atrial appendage closure (LAAC) device within a patient's left atrial appendage (LAA). The assembly includes an LAAC device releasably secured to an LAAC delivery catheter, and a steerable ultrasound catheter rotatably disposed relative to the LAAC delivery catheter.

Alternatively or additionally, the assembly may further include a tubular member, where the LAAC delivery catheter and the steerable ultrasound catheter both extend through the tubular member.

Alternatively or additionally, the steerable ultrasound catheter may be adapted to rotate around the LAAC delivery catheter within the tubular member.

Alternatively or additionally, the steerable ultrasound catheter may be adapted to rotate in place relative to the LAAC delivery catheter within the tubular member.

The above summary of some embodiments is not intended to describe each disclosed embodiment or every implementation of the present disclosure. The Figures, and Detailed Description, which follow, more particularly exemplify these embodiments.

### BRIEF DESCRIPTION OF THE DRAWINGS

The disclosure may be more completely understood in consideration of the following detailed description in connection with the accompanying drawings, in which:

FIG. 1 is a partial cross-sectional view of an LAA (left atrial appendage);

FIG. 2 is a side view of an illustrative left atrial appendage closure (LAAC) device delivery catheter, with the LAAC device shown in a collapsed configuration;

FIG. 3 is a side view of the illustrative LAAC device delivery catheter of FIG. 2, with the LAAC device shown in an expanded configuration;

FIG. 4 is a perspective view of an illustrative expandable framework forming a part of the LAAC device;

FIG. 5 is a perspective view of the illustrative LAAC device;

FIG. 6 is a perspective view of an illustrative assembly including an LAAC device delivery catheter and an ultrasound catheter;

FIG. 7 is a schematic view of a portion of an illustrative assembly including an LAAC device delivery catheter and an ultrasound catheter;

FIGS. 7A and 7B are cross-sectional views of the portion of an illustrative assembly including an LAAC device delivery catheter and an ultrasound catheter;

FIG. 8 is a schematic view of an illustrative assembly including an LAAC device delivery catheter and an ultrasound catheter;

FIG. 9 is a schematic view of an illustrative assembly including an LAAC device delivery catheter and an ultrasound catheter; and

FIG. 10 is a schematic view of an illustrative assembly including an LAAC device delivery catheter including ultrasound transducers disposed on the LAAC device delivery catheter.

While the disclosure is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives falling within the spirit and scope of the disclosure.

### DETAILED DESCRIPTION

The following description should be read with reference to the drawings, which are not necessarily to scale, wherein like reference numerals indicate like elements throughout the several views. The detailed description and drawings are intended to illustrate but not limit the present disclosure. Those skilled in the art will recognize that the various elements described and/or shown may be arranged in various combinations and configurations without departing from the scope of the disclosure. The detailed description and drawings illustrate example embodiments of the disclosure. However, in the interest of clarity and ease of understanding, while every feature and/or element may not be shown in each drawing, the feature(s) and/or element(s) may be understood to be present regardless, unless otherwise specified.

For the following defined terms, these definitions shall be applied, unless a different definition is given in the claims or elsewhere in this specification.

All numeric values are herein assumed to be modified by the term “about,” whether or not explicitly indicated. The term “about” generally refers to a range of numbers that one of skill in the art would consider equivalent to the recited value (i.e., having the same function or result). In many instances, the terms “about” may include numbers that are rounded to the nearest significant figure.

The recitation of numerical ranges by endpoints includes all numbers within that range (e.g. 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise. It is to be noted that in order to facilitate understanding, certain features of the disclosure may be described in the singular, even though those features may be plural or recurring within the disclosed embodiment(s). Each instance of the features may include and/or be encompassed by the singular disclosure(s), unless expressly stated to the contrary. For simplicity and clarity purposes, not all elements of the present disclosure are necessarily shown in each figure or discussed in detail below. However, it will be understood that the following discussion may apply equally to any and/or all of the components for which there are more than one, unless explicitly stated to the contrary. Additionally, not all instances of some elements or features may be shown in each figure for clarity.

Relative terms such as “proximal”, “distal”, “advance”, “retract”, variants thereof, and the like, may be generally considered with respect to the positioning, direction, and/or operation of various elements relative to a user/operator/manipulator of the device, wherein “proximal” and “retract” indicate or refer to closer to or toward the user and “distal” and “advance” indicate or refer to farther from or away from the user. In some instances, the terms “proximal” and “distal” may be arbitrarily assigned in an effort to facilitate understanding of the disclosure, and such instances will be readily apparent to the skilled artisan. Other relative terms, such as “upstream”, “downstream”, “inflow”, and “outflow” refer to a direction of fluid flow within a lumen, such as a body lumen, a blood vessel, or within a device. Still other relative terms, such as “axial”, “circumferential”, “longitudinal”, “lateral”, “radial”, etc. and/or variants thereof generally refer to direction and/or orientation relative to a central longitudinal axis of the disclosed structure or device.

The terms “monolithic” and “unitary” shall generally refer to an element or elements made from or consisting of a single structure or base unit/element. A monolithic and/or unitary element shall exclude structure and/or features made by assembling or otherwise joining multiple discrete elements together.

It is noted that references in the specification to “an embodiment”, “some embodiments”, “other embodiments”, etc., indicate that the embodiment(s) described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it would be within the knowledge of one skilled in the art to use the particular feature, structure, or characteristic in connection with other embodiments, whether or not explicitly described, unless clearly stated to the contrary. That is, the various individual elements described below, even if not explicitly shown in a particular combination, are nevertheless contemplated as being combinable or arrangeable with each other to form other additional embodiments or to complement and/or enrich the described embodiment(s), as would be understood by one of ordinary skill in the art.

For the purpose of clarity, certain identifying numerical nomenclature (e.g., first, second, third, fourth, etc.) may be used throughout the description and/or claims to name and/or differentiate between various described and/or claimed features. It is to be understood that the numerical nomenclature is not intended to be limiting and is exemplary only. In some embodiments, alterations of and deviations from previously used numerical nomenclature may be made in the interest of brevity and clarity. That is, a feature identified as a “first” element may later be referred to as a “second” element, a “third” element, etc. or may be omitted entirely, and/or a different feature may be referred to as the “first” element. The meaning and/or designation in each instance will be apparent to the skilled practitioner.

The following figures illustrate selected components and/or arrangements of an implant for occluding the left atrial appendage, a system for occluding the left atrial appendage, and/or methods of using the implant and/or the system. It should be noted that in any given figure, some features may not be shown, or may be shown schematically, for simplicity. Additional details regarding some of the components of the implant and/or the system may be illustrated in other figures in greater detail. While discussed in the context of occluding the left atrial appendage, the implant and/or the system may also be used for other interventions and/or percutaneous medical procedures within a patient. Similarly, the devices and methods described herein with respect to percutaneous deployment may be used in other types of surgical procedures, as appropriate. For example, in some examples, the devices may be used in a non-percutaneous procedure. Devices and methods in accordance with the disclosure may also be adapted and configured for other uses within the anatomy.

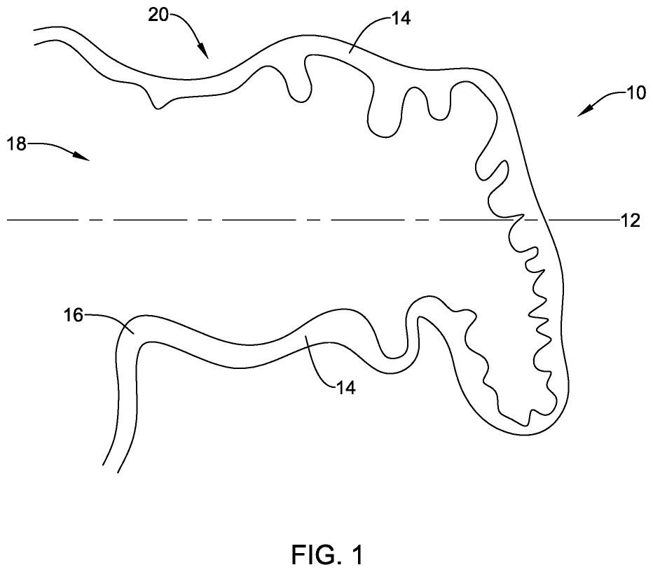

FIG. 1 is a partial cross-sectional view of a left atrial appendage 10. In some embodiments, the left atrial appendage (LAA) 10 may have a complex geometry and/or irregular surface area. It will be appreciated that the illustrated LAA 10 is merely one of many possible shapes and sizes for the LAA 10, which may vary from patient to patient. Those of skill in the art will also recognize that the medical devices, systems, and/or methods disclosed herein may be adapted for various sizes and shapes of the LAA 10, as necessary. The left atrial appendage 10 may include a generally longitudinal axis 12 arranged along a depth of a main body 20 of the left atrial appendage 10. The main body 20 may include a lateral wall 14 and an ostium 16 forming a proximal mouth 18. In some examples, a lateral extent of the ostium 16 and/or the lateral wall 14 may be smaller or less than a depth of the main body 20 along the longitudinal axis 12, or a depth of the main body 20 may be greater than a lateral extent of the ostium 16 and/or the lateral wall 14. In some examples, the LAA 10 may narrow quickly along the depth of the main body 20 or the left atrial appendage may maintain a generally constant lateral extent along a majority of depth of the main body 20. In some examples, the LAA 10 may include a distalmost region formed or arranged as a tail-like element associated with a distal portion of the main body 20. In some examples, the distalmost region may protrude radially or laterally away from the longitudinal axis 12.

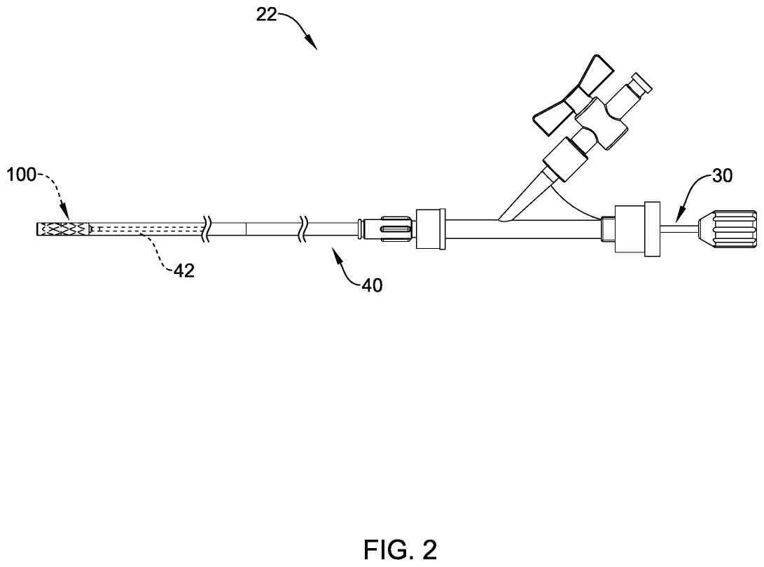

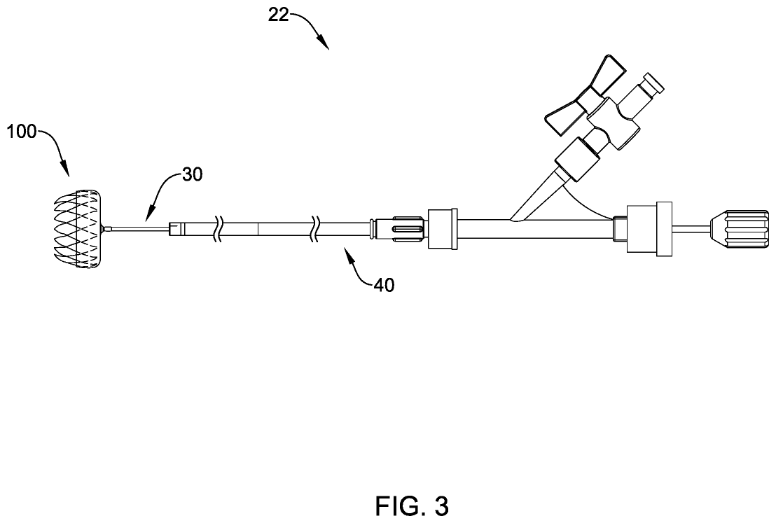

FIGS. 2 and 3 illustrate selected components and/or arrangements of an LAAC device delivery catheter 22 that is adapted to deliver an LAAC device for occluding the LAA 10. It should be noted that in any given figure, some features of the LAAC device delivery catheter 22 may not be shown, or may be shown schematically, for simplicity. Additional details regarding some of the components of the LAAC device delivery catheter 22 may be illustrated in other figures in greater detail.

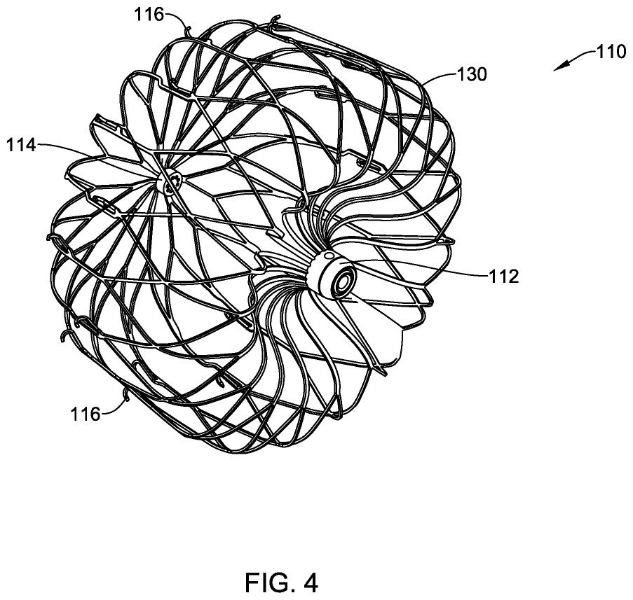

The LAAC device delivery catheter 22 may include a delivery sheath 40 having a lumen 42 extending from a proximal opening to a distal opening, a core wire 30 slidably disposed within the lumen 42, and an LAAC device 100 for occluding the LAA 10. The LAAC device 100 may include an expandable framework 110 (e.g., FIG. 4) configured to shift between a fully constrained configuration (e.g., FIG. 2), wherein the LAAC device 100 is disposed within the lumen 42 proximate the distal opening in the delivery configuration, and a fully unconstrained configuration (e.g., FIG. 3), wherein the LAAC device 100 and/or the expandable framework 110 is configured to shift between the fully constrained configuration and the fully unconstrained configuration as the LAAC device 100 is translated relative to the delivery sheath 40. In at least some embodiments, the expandable framework 110 may be self-biased toward the fully unconstrained configuration.

The LAAC device 100 may be disposed at and/or releasably securable to a distal portion of the core wire 30. The core wire 30 may be slidably and/or rotatably disposed within the lumen 42 of the delivery sheath 40. In some embodiments, a proximal end of the core wire 30 may extend proximally of a proximal end of the delivery sheath 40 and/or the proximal opening of the lumen 42 for manual manipulation by a clinician or practitioner. In some embodiments, the LAAC device 100 may be removably attached, joined, secured, or otherwise connected to a distal end of the core wire 30. The core wire 30 may be configured to and/or may be capable of axially translating the LAAC device 100 relative to the delivery sheath 40. In one example, the core wire 30 may be advanced distally while the delivery sheath 40 is held in a constant position. In another example, the core wire 30 may be advanced distally while the delivery sheath 40 is retracted proximally. In yet another example, the core wire 30 may be held in a constant position while the delivery sheath 40 is retracted proximally relative to the core wire 30 and/or the LAAC device 100. Other configurations are also contemplated. The delivery sheath 40 and/or the core wire 30 may have a selected level of axial stiffness and/or pushability characteristics while also having a selected level of flexibility to permit navigation through the patient's vasculature.

Some suitable, but non-limiting, examples of materials for the LAAC device delivery catheter 22, the core wire 30, the delivery sheath 40, and/or the LAAC device 100, etc. are discussed below. It is contemplated that any exemplary LAAC device disclosed herein may be used in accordance with and/or be associated with the example LAAC device delivery catheter 22 described above.

The LAAC device 100 may include an expandable framework 110 configured to shift axially and/or radially along a central longitudinal axis between the fully constrained configuration and the fully unconstrained configuration. In the fully constrained configuration, the expandable framework 110 may be axially elongated and/or radially compressed. In the fully unconstrained configuration, the expandable framework 110 may be axially shortened and/or radially expanded.

As seen in FIG. 4, which illustrates selected features of the LAAC device 100 in the fully unconstrained configuration, the expandable framework 110 may have a plurality of struts disposed about the central longitudinal axis. In some embodiments, the plurality of struts may define a plurality of cells. In some embodiments, the plurality of cells may be a plurality of closed cells. In some embodiments, the plurality of cells may be a plurality of open cells. In some embodiments, the plurality of cells may include a plurality of open cells and a plurality of closed cells in various combinations and/or arrangements.

The expandable framework 110 may include a proximal hub 112 and a distal hub 114. In some embodiments, the proximal hub 112 and/or the distal hub 114 may be centered on and/or coaxial with the longitudinal axis. The plurality of struts may be joined together at and/or fixedly attached to the proximal hub 112 and/or the distal hub 114. The proximal hub 112 may be configured to releasably connect, secure, and/or attach the LAAC device 100 and/or the expandable framework 110 to the core wire 30. In some embodiments, the proximal hub 112 may include internal threads configured to rotatably and/or threadably engage an externally threaded distal end of the core wire 30. Other configurations for releasably securing the LAAC device 100 to the core wire 30 are also contemplated. As noted herein, some features are not shown in every figure to improve clarity.

The expandable framework 110 and/or the plurality of struts may be formed and/or cut from a tubular member. In some embodiments, the expandable framework 110 and/or the plurality of struts may be integrally formed and/or cut from a unitary member. In some embodiments, the expandable framework 110 and/or the plurality of struts may be integrally formed and/or cut from a unitary tubular member and subsequently formed and/or heat set to a desired shape in the fully unconstrained configuration. In some embodiments, the expandable framework 110 and/or the plurality of struts may be integrally formed and/or cut from a unitary flat member or sheet, and then rolled or formed into a tubular structure and subsequently formed and/or heat set to the desired shape in the fully unconstrained configuration. Some exemplary means and/or methods of making and/or forming the expandable framework 110 and/or the plurality of struts include laser cutting, machining, punching, stamping, electro discharge machining (EDM), chemical dissolution, etc. Other means and/or methods are also contemplated.

As would be understood by the skilled person, anatomical features may vary in size and/or shape. In some embodiments, the left atrial appendage may have an irregular (e.g., elongated and/or oblong) cross-sectional shape. In some embodiments, the expandable framework 110 may be compliant and substantially conform to and/or be in sealing engagement with the shape and/or geometry of a lateral wall of the LAA 10 when deployed and/or expanded therein. In some embodiments, the LAAC device 100 may expand to a size, extent, or shape less than or different from the fully unconstrained configuration, as determined by the surrounding tissue and/or lateral wall of the LAA 10. In some embodiments, the expandable framework 110 may be configured to shape and/or stretch the tissue of the LAA 10 such that the lateral wall of the LAA 10 substantially conforms to an outer shape of the expandable framework 110. Other configurations are also contemplated.

In some embodiments, the expandable framework 110 may include at least one anchoring member 116 extending radially outward therefrom in the fully unconstrained configuration. In some embodiments, the expandable framework 110 may include at least one anchoring member 116 extending radially outward from the expandable framework 110. In some embodiments, the expandable framework 110 may include at least one anchoring member 116 extending radially outward from the expandable framework 110 proximate a proximal shoulder of the expandable framework 110. In some embodiments, the expandable framework 110 may include at least one anchoring member 116 extending radially outward from the expandable framework 110 proximate a midsection of the expandable framework 110. In some embodiments, the at least one anchoring member 116 may be configured to engage with the lateral wall of the main body of the left atrial appendage. In some embodiments, the at least one anchoring member 116 may be formed as J-shaped hooks having a free end extending in and/or directed toward a proximal direction with respect to the central longitudinal axis of the left atrial appendage closure device 100 and/or the expandable framework 110. Other configurations are also contemplated.

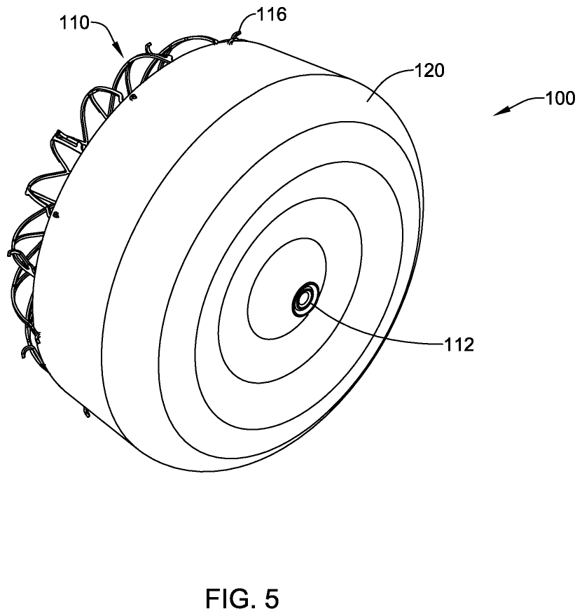

In some embodiments, the LAAC device 100 may optionally include the occlusive element 120 connected to, disposed on, disposed over, disposed about, and/or disposed radially outward of at least a portion of the expandable framework 110 and/or the plurality of struts, as seen in FIG. 5. In some embodiments, the occlusive element 120 may be attached to the proximal hub 112 and/or may be attached to the expandable framework at the proximal hub 112. In some embodiments, the occlusive element 120 may extend radially outward from and/or may extend distally from the proximal hub 112. In some embodiments, the occlusive element 120 may be attached and/or secured to the expandable framework 110 at a plurality of discrete locations. In some embodiments, one of, some of, and/or all of the at least one anchoring member 116 may extend through an occlusive element 120, where present.

In some embodiments, the occlusive element 120 may include a membrane, a fabric, a mesh, a tissue element, or another suitable construction. In some embodiments, the occlusive element 120 may be porous. In some embodiments, the occlusive element 120 may be non-porous. In some embodiments, the occlusive element 120 may be permeable to selected gases and/or fluids. In some embodiments, the occlusive element 120 may be substantially impermeable to selected gases and/or fluids, such as blood, water, etc. In some embodiments, the occlusive element 120 may be designed, sized, and/or configured to prevent thrombus and/or embolic material from passing out of the left atrial appendage into the left atrium and/or the patient's bloodstream. In some embodiments, the occlusive element 120 may be configured to promote endothelization after implantation, thereby effectively removing the target site (e.g., the left atrial appendage, etc.) from the patient's circulatory system. Some suitable, but non-limiting, examples of materials for the occlusive element 120 are discussed below.

The LAAC device delivery catheter 22 discussed above may be used to deliver and deploy the LAAC 100. Because the LAA 10 in one patient is likely different from the LAA 10 in another patient, it will be appreciated that every implantation may not be perfect. Depending on exactly how the LAAC device 100 is deployed relative to the LAA 10, there may be one or more gaps between the LAAC device 100 and the LAA 10. In some cases, leaks, or blood flowing through these one or more gaps, can limit the effectiveness of the LAAC device 100, and are to be minimized if not eliminated. In some cases, ultrasound may be used in combination with the LAAC device delivery catheter 22 in order to look for gaps and/or leaks before the LAAC device delivery catheter 22 is withdrawn. If gaps and/or leaks are found, the LAAC device 100 may be repositioned. Repositioning the LAAC device 100 may include re-sheathing the LAAC device 100 in order to collapse the LAAC device 100 into its collapsed configuration, then repositioning the LAAC device delivery catheter 22 before redeploying the LAAC device 100.

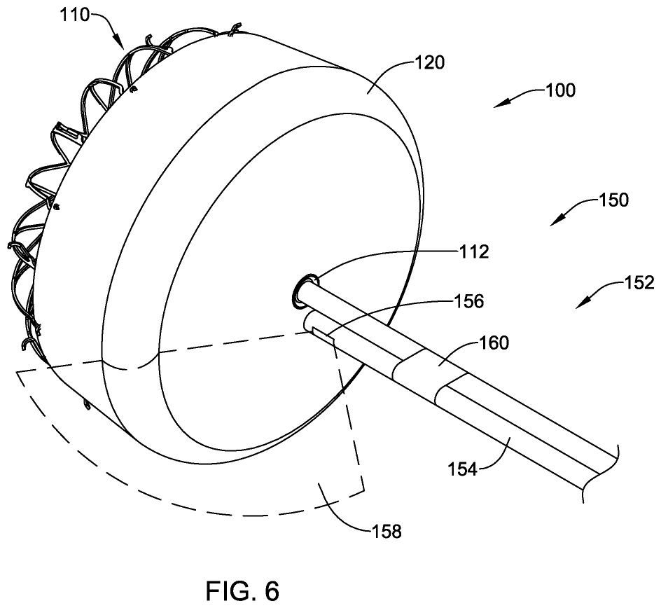

FIG. 6 is a perspective view of an illustrative assembly 150 that includes an LAAC device delivery catheter 152 and an ultrasound catheter 154. In some cases, for example, the ultrasound catheter 154 is an intravascular cardiac echography (ICE) catheter. The assembly 150 includes the LAAC device 100, secured to the LAAC device delivery catheter 152 via the proximal hub 112 of the LAAC device 100. The LAAC device delivery catheter 152 may be considered as being an example of the LAAC device delivery catheter 22, for example. In some cases, the LAAC device delivery catheter 152 may include or otherwise represent the core wire 30 shown with respect to FIGS. 2 and 3. Any features ascribed to the LAAC device delivery catheter 22 may be considered as being applicable to the LAAC device delivery catheter 152.

The ultrasound catheter 154 is shown as including an ultrasound transducer 156. While a single ultrasound transducer 156 is shown, this is merely illustrative, as the ultrasound catheter 154 may include any number of different ultrasound transducers 156. FIG. 6 schematically shows a field 158 that represents what can potentially be seen by the ultrasound transducer 156. It can be seen that the field 158 extends far enough in a radial direction to be able to see beyond a periphery of the LAAC device 100. As a result, this means that the field 158 should extend into any spaces or voids that exist between the LAAC device 100 and the LAA 10 in which the LAACC device 100 is implanted. By rotating the ultrasound transducer 156 in a 360 degree circle, it is possible to see all the way around the LAAC device 100 and thus see any potential gaps and/or leaks that exist. Ultrasound may be able to visualize any gaps and/or leaks that exist between the LAAC device 100 and the LAA 10, and in some cases Doppler may be used for confirmation of possible leaks.

In some cases, gaps may be seen between the LAAC device 100 and the tissue of the LAA 10. In some cases, blood flowing through a gap between the LAAC device 100 and the tissue of the LAA 10 may be visible via ultrasound. If leaks and/or gaps are found, a variety of different actions may be taken. As an example, the physician may decide to implant the LAAC device 100 where the LAAC device 100 currently is, or the physician may decide to reposition the LAAC device 100 before releasing the LAAC device 100. In some cases, the physician may decide to abort the procedure. In some cases, the physician may decide to remove the LAAC device 100 and to implant a different-sized device. In some cases, the physician may decide to add an additional device to resolve the leak, such as adding an embolic coil or a foam plug, for example.

The assembly 150 includes a tubular member 160 that serves to hold in proximity the LAAC device delivery catheter 152 and the ultrasound catheter 154. In some cases, as shown for example in FIG. 6, the tubular member 160 may be a short element that fits snugly around the LAAC device delivery catheter 152 and the ultrasound catheter 154. In some cases, the tubular member 160 may be a longer tubular element, and/or may not fit snugly around the LAAC device delivery catheter 152 and the ultrasound catheter 154. In some cases, the tubular member 160 may be an extruded member having two or more lumens extending therethrough, with the LAAC device delivery catheter 152 extending through one lumen and the ultrasound catheter 154 extending through another lumen. The tubular member 160 may take any of a variety of forms, as along as the ultrasound catheter 154 is able to rotate relative to the LAAC device delivery catheter 152.

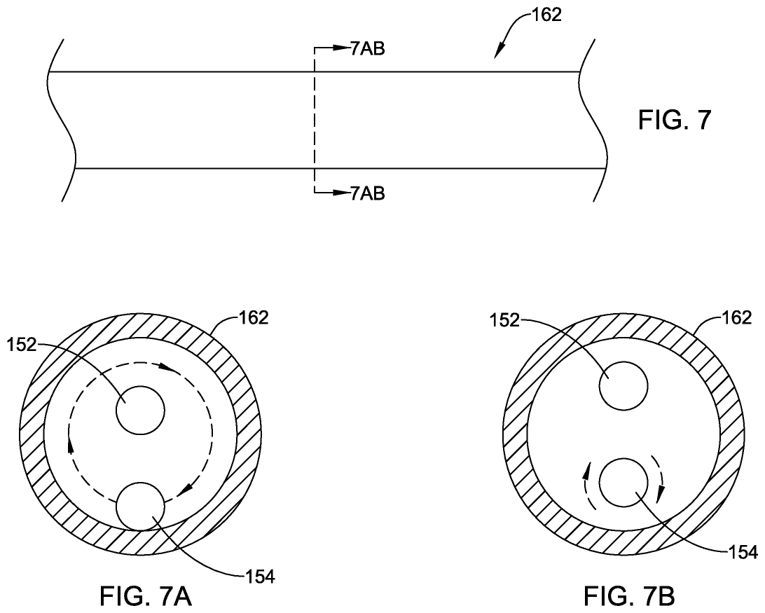

FIG. 7A is a schematic cross-sectional view, showing the LAAC device delivery catheter 152 and the ultrasound catheter 154 inside a tubular member 162. As shown in FIG. 7A, the ultrasound catheter 154 is able to rotate around the LAAC device delivery catheter 152 inside the tubular member 162 with a planetary motion. FIG. 7B is a schematic cross-sectional view showing the LAAC device delivery catheter 152 and the ultrasound catheter 154 inside the tubular member 162. As shown in FIG. 7B, the ultrasound catheter 154 does not rotate around the LAAC device delivery catheter 152, but instead rotates in place next to the LAAC device delivery catheter 152. It will be appreciated that with either form of motion for the ultrasound catheter 154, the ultrasound catheter 154 is able to “see” all the way around the LAAC device 100, apart from regions that may be at least partially blocked by any parts or portions of the LAAC device delivery 152 that may be at least partially radiopaque.

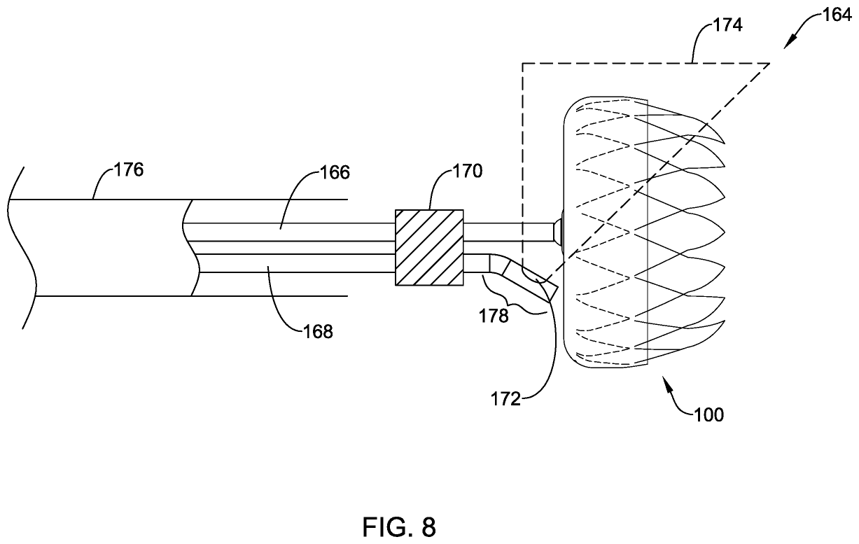

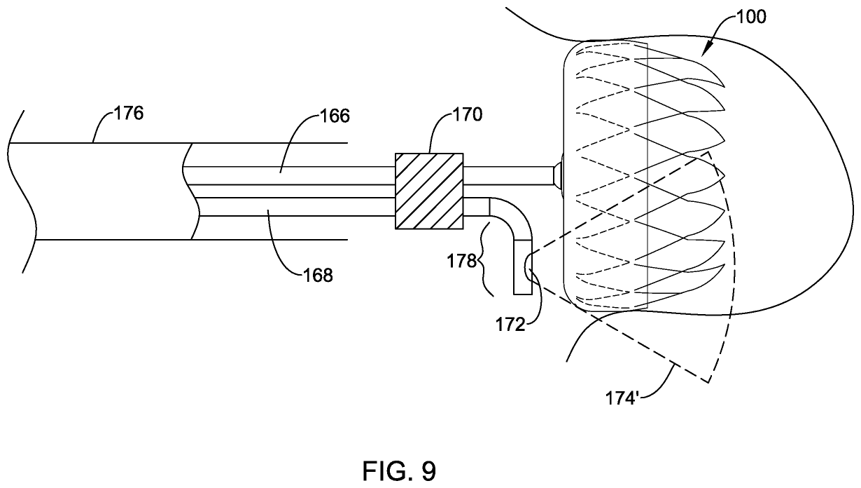

FIGS. 8 and 9 are schematic views of an illustrative assembly 164. The illustrative assembly 164 includes an LAAC device delivery catheter 166 and a steerable ultrasound catheter 168. The LAAC device delivery catheter 166 and the steerable ultrasound catheter 168 are coupled together via a tubular member 170. The steerable ultrasound catheter 168 includes an ultrasound transducer 172. While a single ultrasound transducer 172 is shown, this is merely illustrative, as the steerable ultrasound catheter 168 may include any number of different ultrasound transducers 172. FIGS. 8 and 9 schematically show a field 174 that represents what can potentially be seen by the ultrasound transducer 172. It can be seen that the field 174 extends far enough in a radial direction to be able to see beyond a periphery of the LAAC device 100. As a result, this means that the field 174 should extend into any spaces or voids that exist between the LAAC device 100 and the LAA 10 in which the LAACC device 100 is implanted. By rotating the ultrasound transducer 172 in a 360 degree circle, it is possible to see all the way around the LAAC device 100 and thus see any potential leaks that exist. Ultrasound may be able to visualize any gaps that exist between the LAAC device 100 and the LAA 10, and in some cases Doppler may be used for confirmation. The LAAC device delivery catheter 166 and the steerable ultrasound catheter 168 extend through a guide catheter 176.

The steerable ultrasound catheter 168 has a distal region 178 that can be steered to provide an improved image. In some cases, the field 174 shown in FIG. 8 may not be satisfactory to fully see any gaps that exist between the LAAC device 100 and the LAA 10 in which the LAAC device 100 is implanted. In some cases, curving the distal region 178, which effectively changes a direction in which the ultrasound transducer 172 is aimed, may provide a field 174′ that provides a better view, as shown in FIG. 9. In some cases, the steerable ultrasound catheter 168 may include an elongate member that extends proximally within the steerable ultrasound catheter 168 from the distal region 178, and the elongate member may be manipulated to cause the distal region 178 to bend or curve.

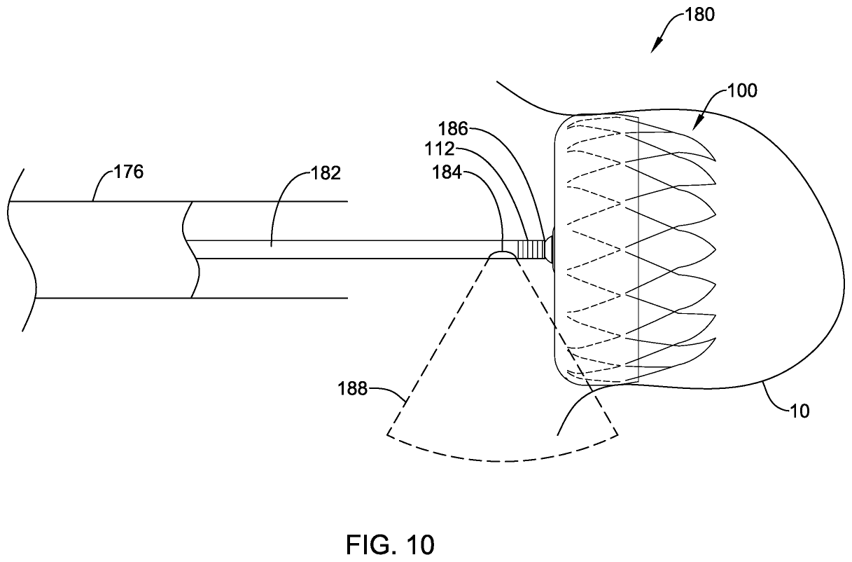

FIG. 10 is a schematic view of an illustrative assembly 180. The illustrative assembly 180 includes a combined catheter 182 that not only has an ultrasound transducer 184 but also has a threaded engagement 186 that is configured to releasable engage the proximal hub 112 of the LAAC device 100. In other words, the combined catheter 182 combines the functionality of the LAAC device delivery catheter and an ultrasound catheter into a unitary device. In some cases, the threaded engagement 186 is adapted to allow for the combined catheter 182 to rotate at least 360 degrees relative to the LAAC device 100 without the LAAC device 100 getting disconnected. As a result, the ultrasound transducer 184, producing a field 188, is able to be rotated at least 360 degrees around without prematurely releasing the LAAC device 100. In some cases, the LAAC device 100 may be intentionally disconnected by rotating the combined catheter 182 multiple revolutions.

While a single ultrasound transducer 184 is shown, this is merely illustrative, as the combined catheter 182 may include any number of different ultrasound transducers 184. By rotating the ultrasound transducer 184 in a 360 degree circle, it is possible to see all the way around the LAAC device 100 and thus see any potential leaks that exist. Ultrasound may be able to visualize any gaps that exist between the LAAC device 100 and the LAA 10, and in some cases Doppler may be used for confirmation.

The materials that can be used for the devices described herein may include those commonly associated with medical devices. The devices described herein, or components thereof, may be made from a metal, metal alloy, polymer (some examples of which are disclosed below), a metal-polymer composite, ceramics, combinations thereof, and the like, or other suitable material. Some examples of suitable metals and metal alloys include stainless steel, such as 304V, 304L, and 316LV stainless steel; mild steel; nickel-titanium alloy such as linear-elastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R30035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; titanium; combinations thereof; and the like; or any other suitable material.

As alluded to herein, within the family of commercially available nickel-titanium or nitinol alloys, is a category designated “linear elastic” or “non-super-elastic” which, although may be similar in chemistry to conventional shape memory and super elastic varieties, may exhibit distinct and useful mechanical properties. Linear elastic and/or non-super-elastic nitinol may be distinguished from super-elastic nitinol in that the linear elastic and/or non-super-elastic nitinol does not display a substantial “superelastic plateau” or “flag region” in its stress/strain curve like super-elastic nitinol does. Instead, in the linear elastic and/or non-super-elastic nitinol, as recoverable strain increases, the stress continues to increase in a substantially linear, or a somewhat, but not necessarily entirely linear relationship until plastic deformation begins or at least in a relationship that is more linear that the super elastic plateau and/or flag region that may be seen with super elastic nitinol. Thus, for the purposes of this disclosure linear elastic and/or non-super-elastic nitinol may also be termed “substantially” linear elastic and/or non-super-elastic nitinol.

In some cases, linear elastic and/or non-super-elastic nitinol may also be distinguishable from super-elastic nitinol in that linear elastic and/or non-super-elastic nitinol may accept up to about 2-5% strain while remaining substantially elastic (e.g., before plastically deforming) whereas super elastic nitinol may accept up to about 8% strain before plastically deforming. Both of these materials can be distinguished from other linear elastic materials such as stainless steel (that can also can be distinguished based on its composition), which may accept only about 0.2 to 0.44 percent strain before plastically deforming.

In some embodiments, the linear elastic and/or non-super-elastic nickel-titanium alloy is an alloy that does not show any martensite/austenite phase changes that are detectable by differential scanning calorimetry (DSC) and dynamic metal thermal analysis (DMTA) analysis over a large temperature range. For example, in some embodiments, there may be no martensite/austenite phase changes detectable by DSC and DMTA analysis in the range of about −60 degrees Celsius (° C.) to about 120° C. in the linear elastic and/or non-super-elastic nickel-titanium alloy. The mechanical bending properties of such material may therefore be generally inert to the effect of temperature over this very broad range of temperature. In some embodiments, the mechanical bending properties of the linear elastic and/or non-super-elastic nickel-titanium alloy at ambient or room temperature are substantially the same as the mechanical properties at body temperature, for example, in that they do not display a super-elastic plateau and/or flag region. In other words, across a broad temperature range, the linear elastic and/or non-super-elastic nickel-titanium alloy maintains its linear elastic and/or non-super-elastic characteristics and/or properties.

In some embodiments, the linear elastic and/or non-super-elastic nickel-titanium alloy may be in the range of about 50 to about 60 weight percent nickel, with the remainder being essentially titanium. In some embodiments, the composition is in the range of about 54 to about 57 weight percent nickel. One example of a suitable nickel-titanium alloy is FHP-NT alloy commercially available from Furukawa Techno Material Co. of Kanagawa, Japan. Some examples of nickel titanium alloys are disclosed in U.S. Pat. Nos. 5,238,004 and 6,508,803, which are incorporated herein by reference. Other suitable materials may include ULTANIUM™ (available from Neo-Metrics) and GUM METAL™ (available from Toyota). In some other embodiments, a superelastic alloy, for example a superelastic nitinol can be used to achieve desired properties.

In at least some embodiments, the devices described herein, or components thereof, may also be doped with, made of, or otherwise include a radiopaque material. Radiopaque materials are understood to be materials capable of producing a relatively bright image on a fluoroscopy screen or another imaging technique during a medical procedure. Some examples of radiopaque materials can include, but are not limited to, gold, platinum, palladium, tantalum, tungsten alloy, polymer material loaded with a radiopaque filler, and the like. Additionally, other radiopaque marker bands and/or coils may also be incorporated into the design of guidewire 10 to achieve the same result.

In some embodiments, a degree of Magnetic Resonance Imaging (MRI) compatibility is imparted into the devices described herein, or components thereof. For example, The devices described herein, or components thereof, may be made of a material that does not substantially distort the image and create substantial artifacts (e.g., gaps in the image). Certain ferromagnetic materials, for example, may not be suitable because they may create artifacts in an MRI image. The devices described herein, or components thereof, may also be made from a material that the MRI machine can image. Some materials that exhibit these characteristics include, for example, tungsten, cobalt-chromium-molybdenum alloys (e.g., UNS: R30003 such as ELGILOY®, PHYNOX®, and the like), nickel-cobalt-chromium-molybdenum alloys (e.g., UNS: R30035 such as MP35-N® and the like), nitinol, and the like, and others.

A sheath or covering (not shown) may be disposed over portions or all of the devices described herein in order to define a generally smooth outer surface. In other embodiments, however, such a sheath or covering may be absent. The sheath may be made from a polymer or other suitable material. Some examples of suitable polymers may include polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane (for example, Polyurethane 85A), polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), MARLEX® high-density polyethylene, MARLEX® low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro(propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, ionomers, biocompatible polymers, other suitable materials, or mixtures, combinations, copolymers thereof, polymer/metal composites, and the like. In some embodiments the sheath can be blended with a liquid crystal polymer (LCP). For example, the mixture can contain up to about 6 percent LCP.

In some embodiments, the exterior surface of the devices described herein may be sandblasted, beadblasted, sodium bicarbonate-blasted, electropolished, etc. In these as well as in some other embodiments, a coating, for example a lubricious, a hydrophilic, a protective, or other type of coating may be applied. Alternatively, a sheath may include a lubricious, hydrophilic, protective, or other type of coating. Hydrophobic coatings such as fluoropolymers provide a dry lubricity which improves guidewire handling and device exchanges. Lubricious coatings improve steerability and improve lesion crossing capability. Suitable lubricious polymers are well known in the art and may include silicone and the like, hydrophilic polymers such as high-density polyethylene (HDPE), polytetrafluoroethylene (PTFE), polyarylene oxides, polyvinylpyrrolidones, polyvinylalcohols, hydroxy alkyl cellulosics, algins, saccharides, caprolactones, and the like, and mixtures and combinations thereof. Hydrophilic polymers may be blended among themselves or with formulated amounts of water insoluble compounds (including some polymers) to yield coatings with suitable lubricity, bonding, and solubility. Some other examples of such coatings and materials and methods used to create such coatings can be found in U.S. Pat. Nos. 6,139,510 and 5,772,609, which are incorporated herein by reference.

Portions of the devices described herein may be formed, for example, by coating, extrusion, co-extrusion, interrupted layer co-extrusion (ILC), or fusing several segments end-to-end. The layer may have a uniform stiffness or a gradual reduction in stiffness from the proximal end to the distal end thereof. The gradual reduction in stiffness may be continuous as by ILC or may be stepped as by fusing together separate extruded tubular segments. The outer layer may be impregnated with a radiopaque filler material to facilitate radiographic visualization. Those skilled in the art will recognize that these materials can vary widely without deviating from the scope of the present disclosure.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The invention's scope is, of course, defined in the language in which the appended claims are expressed.

What is claimed is:

1. A method of implanting a left atrial appendage closure (LAAC) device within a patient's left atrial appendage (LAA), the method comprising:
advancing an assembly to a position proximate the patient's LAA, the assembly including:
an LAAC device releasably secured to an LAAC delivery catheter; and
one or more ultrasound transducers disposed relative to the LAAC delivery catheter;
deploying the LAAC device within the LAA; and
rotating the ultrasound transducers relative to the LAAC device to look for gaps and/or leaks between the LAAC device and the LAA.

2. The method of claim 1, further comprising repositioning the LAAC device in response to detecting gaps and/or leaks.

3. The method of claim 1, wherein the one or more ultrasound transducers are part of an ultrasound catheter.

4. The method of claim 3, wherein the ultrasound catheter is steerable.

5. The method of claim 3, further comprising steering the ultrasound catheter to get a better view of any possible gaps and/or leaks between the LAAC device and the LAA.

6. The method of claim 3, wherein rotating the one or more ultrasound transducers comprises rotating the ultrasound catheter around the LAAC delivery catheter.

7. The method of claim 3, wherein rotating the one or more ultrasound transducers comprises rotating the ultrasound catheter in place relative to the LAAC delivery catheter.

8. The method of claim 1, wherein the one or more ultrasound transducers are disposed on the LAAC delivery device.

9. The method of claim 8, wherein rotating the one or more ultrasound transducers comprises rotating the LAAC delivery device.

10. An assembly adapted for implanting a left atrial appendage closure (LAAC) device within a patient's left atrial appendage (LAA), the assembly comprising:
an LAAC device releasably secured to an LAAC delivery catheter; and
one or more ultrasound transducers disposed relative to the LAAC delivery catheter.

11. The assembly of claim 10, wherein the one or more ultrasound transducers are part of an ultrasound catheter.

12. The assembly of claim 11, wherein the ultrasound catheter is steerable.

13. The assembly of claim 11, wherein the ultrasound catheter is adapted to rotate around the LAAC delivery catheter.

14. The assembly of claim 11, wherein the ultrasound catheter is adapted to rotate in place relative to the LAAC delivery catheter.

15. The assembly of claim 10, wherein the one or more ultrasound transducers are disposed on the LAAC delivery catheter.

16. The assembly of claim 15, wherein the LAAC delivery device has a releasable connection between the LAAC delivery catheter and the LAAC device, and the releasable connection is adapted to permit relative rotation therebetween without releasing the LAAC device.

17. An assembly adapted for implanting a left atrial appendage closure (LAAC) device within a patient's left atrial appendage (LAA), the assembly comprising:
an LAAC device releasably secured to an LAAC delivery catheter; and
a steerable ultrasound catheter rotatably disposed relative to the LAAC delivery catheter.

18. The assembly of claim 17, further comprising a tubular member, where the LAAC delivery catheter and the steerable ultrasound catheter both extend through the tubular member.

19. The assembly of claim 18, wherein the steerable ultrasound catheter is adapted to rotate around the LAAC delivery catheter within the tubular member.

20. The assembly of claim 18, wherein the steerable ultrasound catheter is adapted to rotate in place relative to the LAAC delivery catheter within the tubular member.
